(19) United States
(12) Patent Application Publication
(10) Pub. No.: US 2022/0296870 A1
(43) Pub. Date: Sep. 22, 2022

(54) NON-REMOVABLE CONNECTION SYSTEM

(71) Applicant: GAMBRO LUNDIA AB, Lund (SE)

(72) Inventor: Guillaume VERNIN, Meyzieu (FR)

(21) Appl. No.: 17/634,855

(22) PCT Filed: Aug. 11, 2020
(86) PCT No.: PCT/EP2020/072498

§ 371 Date: Feb. 11, 2022

(30) Foreign Application Priority Data
Aug. 13, 2019 (EP) 19191551.1

(51) Int. Cl. A61M 39/10; A61M 1/36; A61M 60/113; A61M 60/279; A61M 60/37; F16L 37/10; F16L 37/113

(52) U.S. Cl. CPC A61M 39/1011; A61M 1/3661; A61M 60/113; A61M 60/279; A61M 60/37; F16L 37/101; F16L 37/113; A61M 2039/1038

(57) ABSTRACT

Non-removable connection system (100) for a medical apparatus comprising a first connector (1) comprising a fluid coupling portion (2), a second connector (11) comprising a respective fluid coupling portion (12) connectable to the fluid coupling portion (2) of the first connector (1), wherein the second connector (11) comprises a hooking portion (13) and at least one locking projection (14). The non-removable connection system (100) also comprises a collar (20) coupled to the first connector (1) and comprising a respective hooking portion (23) engageable to the hooking portion (13) of the second connector (11) through an engagement rotation (ER), wherein in a engaged configuration the hooking portions (23, 13) prevent axial removal of the collar (20) from the second connector (11). The collar (20) further comprises respective locking projections (24) engageable to the locking projections (14) of the second connector (11) through the engagement rotation; in a coupled configuration the locking projections (24, 14) prevent a disengagement rotation of the collar (20) with respect to the second connector (11).

### FIELD OF THE INVENTION

The present invention relates to a non removable connection system for medical devices particularly configured to be connected to a fluid line, such as a blood line, an infusion line or a monitoring line. In particular said connection system may be connected to the fluid lines of a dialysis machine or may be directly attached to the dialysis machine or to an accessory of the disposable set/dialysis machine. The present invention also refers to a disposable comprising fluid lines of a blood treatment apparatus, such as a dialysis machine, and said connection system.

### BACKGROUND

Common connection systems for medical purposes are already known in the state of the art, for example used to connect fluid lines each other or to a medical machine. In blood treatment apparatuses, such as dialysis machines, connectors are commonly used for connecting fluid lines, for example for connecting in a fluid-tight manner return lines and access lines to respective vascular access systems of the patient, and/or for connecting infusion lines or monitoring lines to the blood or fluid circuit. In blood treatment apparatuses, Luer-type connectors are frequently used. Besides the fact of ensuring the fluid-tight sealing and the suitability to be used in the medical field, connectors are required, in specific cases, to avoid voluntary or involuntary disconnections: for example, where the connector is used on a blood line of a dialysis machine, a disconnection during an operative condition of the machine would lead to dangerous consequences potentially lethal for the patient. Any undesired disconnection may lead to sterility issues and requires the operator intervention, sometimes including the interruption of the treatment.

U.S. Pat. No. 8,882,726 is directed to a drug delivery device defining a reservoir for containing a liquid, such as a medicine. The device comprises a distal end presenting an end-piece traversed by a channel along a longitudinal axis. The end-piece is surrounded by a collar extending from the distal end, wherein the collar is provided with a plurality of outer longitudinal ridges running on the whole circumference of the collar: all the longitudinal ridges have each a sloped surface. The device also comprises a tubular wall having an inner duct, wherein an interface of the tubular wall is associable with the end-piece to define a fluid tight connection. The tubular wall is also provided with an outer flange configured to be engaged with a thread arranged in an inner lateral wall of the collar to allow axial engagement between the tubular wall and the end-piece of the device. The tubular wall also comprises a skirt provided with inner longitudinal projections able to cooperate with the longitudinal ridges and their sloped surfaces, to allow a screwing rotation of the tubular wall and to prevent unscrewing rotation of the same. The applicant points out some drawbacks affecting the device described in U.S. Pat. No. 8,882,726, from manufacturing to ease of use, as well as the impossibility of using it in combination with blood treatment apparatuses. In particular, such connector system would cause twist of the fluid line during connection, determining unwanted restrictions of the fluid line cross section, which might reduce drug or blood flow rate or determine damage of the fluid line itself. Moreover, the architecture of the connector comprises several undercuts and recesses, which makes production very complex and, therefore, expensive.

### OBJECT OF THE INVENTION

The aim of this invention is therefore to at least partially solve one or more of the drawbacks and/or limitations of the previous solutions.

A first object is to provide a non removable connector for medical applications suitable for connecting disposable components, such as fluid lines. An aim is to avoid the need of multiple interventions and checks during the set-up of a medical apparatus and during the following treatment of a patient. A further aim is to reduce the risk for the connection to untighten, either due to human error or to other causes, such as mechanical/thermal stress on the connectors. Another object is to provide a non removable connector easy to produce and cheap. These objects and more, which will appear more from the following description, are substantially achieved by a connection system and a disposable set in accordance with one or more of the following claims and/or aspects.

### SUMMARY SECTION

An aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising a hooking portion (13) and at least one locking member (14);
a collar (20) coupled to the first connector (1) and comprising a respective hooking portion (23) engageable to the hooking portion (13) of the second connector (11) through an engagement rotation along an engagement direction (ER), in a engaged configuration the hooking portion (23) of the collar (20) and the hooking portion (13) of the second connector (11) preventing axial removal of the collar (20) from the second connector (11),
   wherein the collar (20) further comprises a respective at least one locking member (24) engageable to the at least one locking member (14) of the second connector (11) through the engagement rotation along the engagement direction (ER), in a coupled configuration the at least one locking member (24) of the collar (20) and the at least one locking member (14) of the second connector (11) preventing a disengagement rotation of the collar (20) with respect to the second connector (11), said disengagement rotation being a rotation in a opposite direction with respect to said engagement rotation.

A further aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising a hooking portion (13) and at least one locking member (14);
a collar (20) coupled to the first connector (1) and comprising a respective hooking portion (23) engageable to the hooking portion (13) of the second connector (11) through an axial engagement displacement along an engagement direction (ED), in a engaged configuration the hooking portion (23) of the collar (20) and the hooking portion (13) of the second connector (11) preventing axial removal of the collar (20) from the second connector (11),
   wherein the collar (20) further comprises a respective at least one locking member (24) engageable to the at least one locking member (14) of the second connector (11) through the axial engagement displacement of the collar (20) with respect to the second connector (11) along the engagement direction (ED), in a coupled configuration the at least one locking member (24) of the collar (20) and the at least one locking member (14) of the second connector (11) preventing a axial disengagement displacement of the collar (20) with respect to the second connector (11), said axial disengagement displacement being a displacement, in particular a linear displacement, in a opposite direction with respect to said axial engagement displacement.

A further aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising a hooking portion (13) and at least one locking member (14);
a collar (20) coupled to the first connector (1) and comprising a respective hooking portion (23) engageable to the hooking portion (13) of the second connector (11) through at least one between an engagement rotation and an axial engagement displacement along an engagement direction (ER, ED), in an engaged configuration the hooking portion (23) of the collar (20) and the hooking portion (13) of the second connector (11) preventing axial removal of the collar (20) from the second connector (11),
   wherein the collar (20) further comprises a respective at least one locking member (24) engageable to the at least one locking member (14) of the second connector (11) through at least one between the engagement rotation and the axial engagement displacement of the collar (20) with respect to the second connector (11) along the engagement direction (ER, ED), in a coupled configuration the at least one locking member (24) of the collar (20) and the at least one locking member (14) of the second connector (11) preventing at least one between a disengagement rotation and an axial disengagement displacement of the collar (20) with respect to the second connector (11), said disengagement rotation being a rotation in a opposite direction with respect to said engagement rotation and the axial disengagement displacement being a displacement, in particular a linear displacement, in a opposite direction with respect to said axial engagement displacement.

A further aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising at least one locking member (14);
a collar (20) coupled to the first connector (1) and comprising respective at least one locking member (24) engageable to the at least one locking member (14) of the second connector (11) through an axial engagement displacement along an engagement direction (ED), in a coupled configuration the at least one locking member (24) of the collar (20) and the at least one locking member (14) of the second connector (11) preventing an axial disengagement displacement of the collar (20) with respect to the second connector (11),
   said axial disengagement displacement being a displacement, in particular a linear displacement, in an opposite direction with respect to said axial engagement displacement.

In an aspect according to the preceding aspect, the second connector (11) comprises a hooking portion (13), and the collar comprises a respective hooking portion (23) engageable to the hooking portion (13) of the second connector (11) through an engagement rotation along an engagement direction (ER), in a engaged configuration the hooking portion (23) of the collar (20) and the hooking portion (13) of the second connector (11) preventing axial removal of the collar (20) from the second connector (11).

A further aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising a hooking portion (13) and at least one locking projection (14);
a collar (20) coupled to the first connector (1) and comprising a respective hooking portion (23) engageable to the hooking portion (13) of the second connector (11) through an axial engagement displacement along an engagement direction (ER), in a engaged configuration the hooking portion (23) of the collar (20) and the hooking portion (13) of the second connector (11) preventing axial removal of the collar (20) from the second connector (11),
   wherein the collar (20) further comprises a respective at least one locking projection (24) engageable to the at least one locking projection (14) of the second connector (11) through the axial engagement displacement of the collar (20) with respect to the second connector (11) along the engagement direction (ER), in a coupled configuration the at least one locking projection (24) of the collar (20) and the at least one locking projection (14) of the second connector (11) preventing an axial disengagement displacement of the collar (20) with respect to the second connector (11), said disengagement displacement being a displacement, in particular a linear displacement, in a opposite direction with respect to said axial engagement displacement.

A first aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising a hooking portion (13) and at least one locking projection (14);
a collar (20) coupled to the first connector (1) and comprising a respective hooking portion (23) engageable to the hooking portion (13) of the second connector (11) through an engagement rotation along an engagement direction (ER), in a engaged configuration the hooking portion (23) of the collar (20) and the hooking portion (13) of the second connector (11) preventing axial removal of the collar (20) from the second connector (11),
   wherein the collar (20) further comprises a respective at least one locking projection (24) engageable to the at least one locking projection (14) of the second connector (11) through the engagement rotation of the collar (20) with respect to the second connector (11) along the engagement direction (ER), in a coupled configuration the at least one locking projection (24) of the collar (20) and the at least one locking projection (14) of the second connector (11) preventing a disengagement rotation of the collar (20) with respect to the second connector (11), said disengagement rotation being a rotation in a opposite direction with respect to said engagement rotation.

In a 2^{nd} aspect according to any one of the preceding aspects, the connection system (100) is configurable, through the engagement rotation, in an unlocked condition, wherein the collar (20) is movable by rotation with respect to the first connector (1) and the at least one locking projection or locking member (24) of the collar (20) and the at least one locking projection or locking member (14) of the second connector (11) are in an uncoupled configuration.

In a 2^{nd} aspect according to any one of the preceding aspects, the connection system (100) is configurable, through the axial engagement displacement, in an unlocked condition, wherein the at least one locking projection or locking member (24) of the collar (20) and the at least one locking projection or locking member (14) of the second connector (11) are in an uncoupled configuration, and optionally the collar (20) is movable by rotation with respect to the first connector (1).

In a 3^{rd} aspect according to any one of the preceding aspects, the connection system (100) is configurable, through the engagement rotation, in a locked condition, wherein the at least one locking projection (24) of the collar (20) and the at least one locking projection (14) of the second connector (11) are in the coupled configuration, and the hooking portion (23) of the collar (20) and the hooking portion (13) of the second connector (11) are in the engaged configuration, in said locked condition the collar (20) being irremovably engaged to the second connector (11) through said engagement rotation and the at least one locking projection (14) and the hooking portion (13) of the second connector (11) respectively cooperating with the at least one locking projection (24) and the hooking portion (23) of the collar (20) preventing disengagement, in particular axial disengagement, of the first connector (1) from the second connector (11).

In a 3^{rd}a aspect according to any one of the preceding aspects, the connection system (100) is configurable, through the axial engagement displacement, in a locked condition, wherein the at least one locking projection or member (24) of the collar (20) and the at least one locking projection or locking member (14) of the second connector (11) are in the coupled configuration, and the hooking portion (23) of the collar (20) and the hooking portion (13) of the second connector (11) are in the engaged configuration, in said locked condition the collar (20) being irremovably engaged to the second connector (11) through said axial engagement displacement and the at least one locking projection or member (14) and the hooking portion (13) of the second connector (11) respectively cooperating with the at least one locking projection or member (24) and the hooking portion (23) of the collar (20) preventing disengagement, in particular axial disengagement, of the first connector (1) from the second connector (11).

In a 3^{rd}b aspect according to any one of the preceding aspects, the connection system (100) is configurable, through the axial engagement displacement, in a locked condition, wherein the at least one locking projection or member (24) of the collar (20) and the at least one locking projection or member (14) of the second connector (11) are in the coupled configuration, in said locked condition the collar (20) being irremovably engaged to the second connector (11) through said axial engagement displacement, and the at least one locking projection or member (14) of the second connector (11) cooperating with the at least one locking projection or member (24) preventing disengagement, in particular axial disengagement, of the first connector (1) from the second connector (11).

In a further aspect according to any one of the preceding aspects, a switch from the locked condition to the unlocked condition is prevented by the at least one locking projection or locking member (14, 24).

In a 4th aspect according to any one of the preceding aspects, the first connector (1), in particular the coupling portion (2) of the first connector (1), extends in length along a fluid flow axis, and the second connector (11), in particular the coupling portion (12) of the second connector (11), extends in length along a respective fluid flow axis, wherein the fluid flow axis of the first connector (1) being aligned, at least when the coupling portion (2) of the first connector (1) is connected to the coupling portion (12) of the second connector (11), to the fluid flow axis of the second connector (11).

In a 5th aspect according to any one of the preceding aspects, the engaged configuration determines an axial constraint between the first and the second connectors (1, 11), in particular said axial constraint being along the fluid flow axis of the first connector (1) and/or of the second connector (11).

In a 6th aspect according to any one of the preceding aspects, the coupled configuration is defined at least at an end stage of the engagement rotation between the collar (20) and the second connector (11).

In a 7th aspect according to any one of the preceding aspects, the first connector (1) comprises a main abutment portion (5) and the collar (20) comprises a respective abutment portion (25) configured to contact the main abutment portion (5) of the first connector (1) when disposed in an abutment configuration.

In a 8th aspect according to the previous aspect, wherein, in said abutment configuration, the collar (20) is axially constrained with respect to the first connector (1) at least along one axial direction, so that, at least when the connection system (100) is disposed in the engaged configuration, the collar (20) axially engages the first connector (1) to the second connector (11), in particular said axial direction being parallel, optionally coincident, to the fluid flow axis of the first connector (1).

In a 9th aspect according to any one of the preceding aspects, the first connector comprises an auxiliary abutment portion (6), said auxiliary abutment portion being configured to block an axial sliding of the collar (20) along a direction opposite to said axial direction.

In a 10th aspect according to any one of the preceding aspects, the collar (20) is:
axially constrained to the first connector (1) through said main and auxiliary abutment portions (5, 6) of the first connector (1), so that an axial movement of the collar (20) with respect to the first connector (1) is substantially prevented; or
axially movable with respect to the first connector within an axial gap defined between said main and auxiliary abutment portions (5, 6) of the first connector (1), and axially constrained to the first connector (1) out of said axial gap, in particular said axial movement being along the fluid flow axis of the first connector (1) and/or of the second connector (11).

In an 11th aspect according to any one of the preceding aspects, the first connector (1) comprises a main body having tubular shape defining an internal channel (7) for passage of the fluid.

In a 12th aspect according to any one of the preceding aspects, said main and auxiliary abutment portions (5, 6) radially extend away from said main body.

In a 13th aspect according to any one of the preceding aspects, the first connector (1) further comprising a recess (8) interposed between the main and the auxiliary abutment portions (5, 6).

In a 14th aspect according to any one of the preceding aspects, the abutment portion (25) of the collar (20) is arranged between the main and the auxiliary abutment portions (5, 6) of the first connector (1) into said recess (8).

In a 15th aspect according to any one of the preceding aspects, the collar (20) is axially movable with respect to the first connector (1) of an amount proportional to the distance between the main and the auxiliary abutment portions (5, 6) of the first connector (1).

In a 16th aspect according to any one of the preceding aspects, the fluid coupling portion (2) of the first connector (1) includes a tip portion (2a) defining an internal channel (7) for fluid transport, in particular said tip portion (2a) having a tapered external surface.

In a 17th aspect according to any one of the preceding aspects, the fluid coupling portion (12) of the second connector (11) includes a terminal portion (12a) defining an internal channel (17) for fluid transport.

In a 18th aspect according to any one of the preceding aspects, said terminal portion (12a) of the second connector (11) has a tapered internal surface optionally counter shaped to the tapered external surface of the tip portion (2a) of the first connector.

In a 19th aspect according to any one of the preceding aspects, at least part of the tip portion (2a) of the first connector (1) is configured to enter into the terminal portion (12a) of the second connector (11), in particular said tapered external surface coupling to said tapered internal surface to connect the first and the second connectors (1, 11) in a fluid tight manner.

In a 19^{th}a aspect according to any one of the preceding aspects, the first connector (1) abuts against the second connector (11) at least in the locked condition.

In a 19^{th}b aspect according to any one of the preceding aspects, the external surface of the first connector (1) abuts against said tapered internal surface of the second connector (11) at least in the locked condition.

In a 20th aspect according to any one of the preceding aspects, the collar (20) comprises a main body having tubular shape extending between a first opening (21) configured to receive the second connector (11), in particular to receive the coupling portion (12), the hooking portion (13) and the at least one locking projection (14) of the second connector (11), and a second opening (22) receiving by insertion the first connector (1).

In a 21st aspect according to any one of the preceding aspects, the collar surrounds externally at least part of the first connector (1) and the main body of the collar (20) being coaxial with the first connector (1).

In a 22nd aspect according to any one of the preceding aspects, the second opening (22) is arranged at a recess (8) of the first connector (1) to allow coupling.

In a 23rd aspect according to any one of the preceding aspects, an abutment portion (25) of the collar (20) extends radially inwardly is arranged at the second opening (22).

In a 24th aspect according to any one of the preceding aspects, said at least one locking projection or member (24) of the collar (20) is arranged at an inlet of the first opening (21).

In a 25th aspect according to any one of the preceding aspects, said hooking portion (23) of the collar (20) is interposed between said at least one locking projection (24) and the second opening (22) of the collar (20).

In a 26th aspect according to any one of the preceding aspects, the hooking portion (13) of the second connector (11) is interposed between the at least one locking projection (14) and a terminal portion (12a) of the coupling portion (12) of the second connector (11).

In a 27th aspect according to any one of the preceding aspects, said hooking portion (23) of the collar (20) is arranged at an inlet of the first opening (21), said at least one locking projection (24) of the collar (20) being interposed between said hooking portion (23) and the second opening (22) of the collar (20); and
the at least one locking projection or member (14) of the second connector (11) is interposed between the hooking portion (13) and a terminal portion (12a) of the coupling portion (12) of the second connector (11).

In a 28th aspect according to any one of the preceding aspects, the hooking portion (23) of the collar (20) and the hooking portion (13) of the second connector both comprise respective threads configured to engage each other, in particular through the engagement rotation, to define the engaged configuration; or respective undercut joints configured to engage each other as a bayonet coupling, in particular through the engagement rotation, to define the engaged configuration.

In a 29th aspect according to any one of the preceding aspects, the collar (20) comprises a main body having tubular shape extending in depth between an internal lateral wall (20a) and an external lateral wall (20b).

In a 30th aspect according to any one of the preceding aspects, the collar (20) is mounted over the first connector (1) so that the main body externally surrounds at least part of the first connector (11), in particular surrounding at least part of the coupling portion (2) of the first connector (1).

In a 31st aspect according to any one of the preceding aspects, said hooking portion (23) of the collar (20), in particular said threads of the collar (20), is arranged on said internal lateral wall (20a) of the collar (20).

In a 32nd aspect according to any one of the preceding aspects, the at least one locking projection (24) of the collar (20) is radially arranged on the same internal lateral wall (20a) of the collar (20).

In a 32nd aspect according to any one of the preceding aspects, said hooking portion (23) of the collar is side by side with said at least one locking projection (24) of the collar (20).

In a 33th aspect according to any one of the preceding aspects, the second connector (11) has tubular shape defining an internal channel (17) for fluid transport, said tubular shape of the second connector (11) comprising an external lateral wall (11a).

In a 34th aspect according to any one of the preceding aspects, said hooking portion (13) of the second connector (11), in particular said threads of the second connector (11), is arranged on said external lateral wall (11a) of the second connector (11), in particular said hooking portion (13) being arranged at the coupling portion (12) of the second connector (11).

In a 35th aspect according to any one of the preceding aspects, the at least one locking projection (14) of the second connector (11) being radially arranged on said external lateral wall (11a) of the second connector (11), in particular said at least one locking projection (14) being arranged at the coupling portion (12) of the second connector (11).

In a 36th aspect according to any one of the preceding aspects, in particular said hooking portion (13) of the second connector (11) is side by side with said at least one locking projection (14) of the second connector (11).

In a 37th aspect according to any one of the preceding aspects, both the at least one locking projection (14) of the second connector (11) and the at least one locking projection (24) of the collar (20) comprise respectively one or more socket teeth, in particular a plurality of socket teeth, in particular said socket teeth being wedge-shaped or having a saw tooth shape.

In a 38th aspect according to any one of the preceding aspects, when the socket teeth of the collar (20) face, in particular contact, the socket teeth of the second connector (11), the engagement rotation is allowed and the disengagement rotation is prevented.

In a 39th aspect according to any one of the preceding aspects, the socket teeth of both the second connector (11) and the collar (20) radially define recesses and protrusions alternately.

In a 40th aspect according to any one of the preceding aspects, the second connector (11) defines a maximum external diameter of encumbrance measured at the locking projections (14), in particular at the protrusions of the one or more socket teeth, wherein the collar (20) has tubular shape and has a minimum internal diameter measured at the locking projections (24), in particular at the protrusions of the socket teeth of the collar (20).

In a 41st aspect according to the previous aspect, said maximum external diameter of encumbrance being higher than said minimum internal diameter so that, when the socket teeth of the second connector (11) are faced to, in particular in contact with, the socket teeth of the collar (20), the protrusions of the socket teeth of the second connector (11) are inserted into the recesses of the socket teeth of the collar (20) and vice versa.

In a further aspect according to any one of the preceding aspects, the at least one locking member (14) of the second connector comprises at least one recess, optionally an aperture, and the at least one locking member (24) of the collar (20) comprises at least one protrusion, the at least one protrusion of the collar (20) being configured in the locked condition to engage the recess of the second connector (11) to define an non removable axial engagement, in particular to enter at least partially into said recess, in particular said protrusion radially extending inwardly.

In a further aspect according to any one of the preceding aspects, the at least one locking member (14) of the second connector comprises at least one protrusion, and the at least one locking member (24) of the collar (20) comprises at least one recess, optionally an aperture, the at least one protrusion of the second connector (11) being configured in the locked condition to engage the recess of the collar (20) to define an non removable axial engagement, in particular to enter at least partially into said recess, in particular said protrusion radially extending outwardly.

In a further aspect according to any one of the preceding aspects, both the at least one locking projection or member (14) of the second connector (11) and the at least one locking projection or member (24) of the collar (20) comprise at least one protrusion respectively radially extending outwardly and inwardly, the protrusions of the collar (20) comprising an abutment portion (24c) facing an inner volume of the collar (20) and the protrusions of the second connector (11) comprising an abutment portion (14c) facing opposite with respect to the fluid coupling portion (12) of the second connector (11), the abutment portion (24c) of the protrusions (24) of the collar (20) abutting against the abutment portion (14c) of the protrusions (14) of the second connector (11) when the connection system is arranged in the locked condition to prevent axial disengagement of the collar from the second connector.

In an aspect according to the preceding aspect, the protrusions of the collar (20) define a minimum internal diameter lower and the protrusions of the second collar define a maximum external diameter, said minimum internal diameter being lower than said maximum external diameter.

In a further aspect according to any one of the preceding aspects, the collar (20) comprises one or more fingers (26) arranged at the first opening (21) and in particular extending substantially parallel to the axis of the collar (20), in particular the collar comprising a plurality of fingers arranged at the first opening angularly distributed about the axis of the collar, said fingers (26) carrying the at least one locking projections or member (24) of the collar (20), in particular carrying the protrusions of the collar (20).

In an aspect according to the preceding aspect, the locking projections or member (24) of the collar define a minimum inner diameter smaller than a maximum outer diameter defined by the locking projections or members (14) of the second connector (11), wherein the said fingers (26) of the collar (20) are configured to radially flex outwardly during coupling of the locking projections (14) of the second connector with the locking projections (24) of the collar (20).

In a 42nd aspect according to any one of the preceding aspects, the first connector is a male connector and the second connector is a female connector.

In a 43th aspect according to any one of the preceding aspects, at least when the connection system (100) is disposed in the engaged configuration, at least part of the second connector (11), in particular at least part of the coupling portion (12) of the second connector (11), is interposed between the collar (20) and at least part of the first connector (1), in particular interposed between the collar (20) and at least part of the coupling portion (2) of the first connector (1).

In a 44th aspect according to any one of the preceding aspects, both the first and the second connectors (1, 11) are Luer type connectors.

In a 45th aspect according to any one of the preceding aspects, the first connector (1) extends in length between its coupling portion (2) and a connection port (9) configured to be connected to a fluid line (51), such as a blood circuit (50a) or an infusion line (54) or a monitoring line (55) of a medical machine (200), in particular a blood treatment apparatus, more in particular a dialysis machine.

In a 46th aspect according to any one of the preceding aspects, the second connector (11) extends in length between its coupling portion (12) and a connection port (19) configured to be connected to a fluid line (51), such as a blood circuit (50a) or an infusion line (54) or a monitoring line (55) of a medical machine (200), in particular a blood treatment apparatus, more in particular a dialysis machine.

In a 47th aspect according to any one of the preceding aspects, the connection system (100) comprises a fluid line (51), said connection port (9) being fixed to an end of said fluid line (51), such as a blood circuit (50a) or an infusion line (54) or a monitoring line (55) of a medical machine (200), in particular a blood treatment apparatus, more in particular a dialysis machine, or said connection port (9) of the first connector (1) being fixed to the medical machine (200) directly.

In a 48th aspect according to any one of the preceding aspects, the connection system (100) comprises a fluid line (51), wherein the second connector (11) extends in length between its coupling portion (12) and a respective connection port (19), said connection port (19) being fixed to an end of said fluid line (51), such as a tube, in particular a plastic tube, of a medical machine (200), in particular a blood treatment apparatus, more in particular a dialysis machine, or said connection port (19) of the second connector (11) being fixed to the medical machine (200) directly.

In a 49th aspect according to any one of the preceding aspects, said fluid line (51) is a blood circuit (50a) or an infusion line (54) or a monitoring line (55) of a disposable set (50) of a blood treatment apparatus (200), such as a dialysis machine.

In a 50th aspect according to any one of the preceding aspects, the collar (20) is coaxial to the first connector (1).

In a 51st aspect according to any one of the preceding aspects, the first connector (1) and the collar (20) are made in two separate pieces subsequently axially coupled, the collar (20) being free to rotate with respect to the first connector (1) at least when the second connector (11) is not coupled to the collar (20) and to the first connector (1).

In a further aspect according to any one of the preceding aspects (but aspect 51), the first connector (1) and the collar (20) are made in one piece, in particular the collar (20) being both axially and rotationally coupled to the first connector (1).

In a 52nd aspect according to any one of the preceding aspects, the first connector (1) is made of plastic material.

In a 53rd aspect according to any one of the preceding aspects, the collar (20) and/or the second connector (11) is made of plastic material.

In a 54th aspect according to any one of the preceding aspects, the locking portion (14) of the second connector (11) and/or the locking portion (24) of the collar (20) have a width, measured parallel to the fluid flow axis, higher than a thread pitch of the hooking portion of the collar (20) and/or than a thread pitch of the hooking portion of the second connector (11), particularly in order to prevent decoupling.

In a 55th aspect according to any one of the preceding aspects, the first connector (1), the second connector (11) and the collar (20) are made of the same material.

A 56th aspect refers to a non-removable connection system (100) for a medical apparatus, said connection system (100) comprising:
a first connector (1) comprising a fluid coupling portion (2);
a second connector (11) comprising a respective fluid coupling portion (12) connectable, in a fluid tight manner, to the fluid coupling portion (2) of the first connector (1), so that a fluid is able to internally flow between the first and the second connector (1, 11), the second connector (11) further comprising a hooking portion (13) and at least one locking projection (14);
a collar (20) coupled to the first connector (1) and comprising a respective hooking portion (23) engageable to the hooking portion (13) of the second connector (11) through an engagement rotation along an engagement direction (ER), in a engaged configuration the hooking portion (23) of the collar (20) and the hooking portion (13) of the second connector (11) preventing axial removal of the collar (20) from the second connector (11),
   wherein the collar (20) further comprises a respective at least one locking projection (24) engageable to the at least one locking projection (14) of the second connector (11) through the engagement rotation of the collar (20) with respect to the second connector (11) along the engagement direction (ER), in a coupled configuration the at least one locking projection (24) of the collar (20) and the at least one locking projection (14) of the second connector (11) preventing a disengagement rotation of the collar (20) with respect to the second connector (11), said disengagement rotation being a rotation in a opposite direction with respect to said engagement rotation;

wherein through the engagement rotation, the connection system (100) is configurable in a locked condition, wherein the at least one locking projection (24) of the collar (20) and the at least one locking projection (14) of the second connector (11) are in the coupled configuration, and the hooking portion (23) of the collar (20) and the hooking portion (13) of the second connector (11) are in the engaged configuration, in said locked condition the collar (20) being irremovably engaged to the second connector (11) through said engagement rotation and the at least one locking projection (14) and the hooking portion (13) of the second connector (11) respectively cooperating with the at least one locking projection (24) and the hooking portion (23) of the collar (20) preventing disengagement of the first connector (1) from the second connector (11), wherein the collar (20) has a tubular shape extending between an internal lateral wall (20a) and an external lateral wall (20b), both the hooking portion (23) and the at least one locking projection (24) being arranged on said internal lateral wall (20a).

In a 57th aspect according to the previous aspect, said non-removable connection system (100) is according to any one of the aspects from 1 to 55.

A 58th aspect refers to a disposable set (50) for a blood treatment apparatus, said disposable set comprising:
a blood circuit (50a) including:
   an access line (52) configured to be connected to a patient (300) and to an inlet port (201a) of a blood treatment unit (201);
   a return line (53) configured to be connected to the patient (300) and to an outlet port (201b) of the blood treatment unit (201).

In a 59th aspect according to the previous aspect, said disposable (50) set comprises:
an infusion line (54) connected to the blood circuit (52) and/or configured to be connected to the patient (300).

In a 60th aspect according to any one of the preceding aspects 58 or 59, the disposable set (50) comprises a monitoring line (55) connected to the blood circuit (50a) or to the infusion line (54).

In a 61st aspect according to any one of the preceding aspects 58 or 59 or 60, the disposable set (50) comprises at least one connection system (100) according to any of the aspects from 1 to 57 connected to at least one between the access line (52), the return line (53), the infusion line (54) and the monitoring line (55).

In a 62nd aspect according to any one of the preceding aspects from 58 to 61, the infusion line (54) comprises a calcium solution container (60b), wherein the infusion line (54) includes one end connected to the calcium concentrated solution container (60b), the connection system (100) being coupled to the infusion line (54), for example one of the first and second connectors (1, 11) being connected to said one end, the other of said first and second connectors (1, 11) being connected to the calcium solution container (60b).

In a 63rd aspect according to any one of the preceding aspects from 58 to 62, the calcium solution container (60b) is a syringe, an end of the syringe including, or being connected to, one of the first and second connectors (1, 11).

In a 64th aspect according to any one of the preceding aspects from 58 to 63, the infusion line (54) comprises a citrate solution container (60a), wherein the infusion line (54) includes one end connected to the citrate concentrated solution container (60a), one of the first and second connectors (1, 11) being connected to said one end, the other of said first and second connectors (1, 11) being connected to the citrate solution container (60a).

In a 65th aspect according to any one of the preceding aspects from 58 to 64, the citrate solution container (60a) is a bag, an end of said bag including or being connected to one of the first and second connectors (1, 11).

In a 66th aspect according to any one of the preceding aspects from 58 to 64, the disposable set comprises a blood warmer accessory (70) connected to the blood circuit (50a), particularly placed on the return line, an inlet (70a) and/or an outlet (70b) of said blood warmer accessory (70) being connected to the blood circuit (50a) through said at least one connection system (100).

In a 67th aspect according to any one of the preceding aspects from 58 to 66, the disposable set comprises the infusion line (54) connected to the return line (53), particularly through said at least one connection system (100).

In a 68th aspect according to any one of the preceding aspects from 58 to 67, the disposable set comprises the monitoring line (55) connected to the blood circuit through said at least one connection system (100), said monitoring line (55) being configured to be connected to a pressure or temperature gauge or to be used as a site access for blood sampling.

In a 69th aspect according to any one of the preceding aspects from 58 to 68, the disposable set comprises an auxiliary disposable accessory (80), such as a gas exchanger, an oxygenator, an auxiliary fluid arrangement, optionally the accessory being connected to the blood circuit (50a) through the connection system (100).

In a 70th aspect according to any one of the preceding aspects from 58 to 69, the disposable set comprises an add-on accessory (81) interposed between the patient vascular access and the blood circuit, the add-on accessory (81) being connected to the blood circuit (50a) through the connection system (100).

A 71st aspect refers to a method of the connection system (100) optionally according to any of the preceding aspects from 1 to 57, said connection method comprising the steps of:
arrange the first connector (1) and the collar (20);
arrange the second connector (11) separated from the first connector (1) and the collar (20);
approach the hooking portion (13) of the second connector (11) to the hooking portion (23) of the collar (20), in particular wherein the coupling portion (2) of the first connector (1) is aligned to the coupling portion (12) of the second connector (11);
rotate the collar (20) with respect to both the second connector (11) along the engagement direction of the engagement rotation to determine both the engaged configuration and the coupled configuration of the connection system (100), defining the locked condition.

In a 72^{nd} aspect according to any one of the preceding aspects, the engagement rotation is defined by a relative rotation between the collar (20) and the second connector (11).

In a 73^{rd} aspect according to any one of the preceding aspects, the engagement rotation is defined by a relative rotation between the collar (20) and the first connector (1).

In a 74^{th} aspect according to any one of the preceding aspects, the engagement rotation is defined by a relative rotation between the collar (20) and both the first and the second connectors (1, 11), the first and the second connectors (1, 11) being rotationally coupled each other.

In a 75^{th} aspect according to any one of the preceding aspects, in the coupled configuration, and in particular in the locked condition, a rotation of the collar (20) with respect to the second connector (11) is prevented, in particular said prevented rotation being in a direction opposite to the engagement direction.

In a 76^{th} aspect according to any one of the preceding aspects, in the coupled configuration, and in particular in the locked condition, a rotation of the collar (20) with respect to the first connector (1) is prevented, in particular said prevented rotation being in a direction opposite to the engagement direction.

In a 77^{th} aspect according to any one of the preceding aspects, in the locked condition the first connector (1), the second connector (11) and the collar (20) are rotationally coupled or fixed each other defining a single rigid body.

In a 78^{th} aspect according to any one of the preceding aspects, in the locked condition relative movements, in particular axial and rotational movements, between the first connector (1), the second connector (11) and the collar (20) are prevented.

In a 79^{th} aspect according to any one of the preceding aspects, the axial engagement displacement is a displacement directed substantially parallel to the axis of the first and second connectors, in particular said axial engagement displacement being a displacement directed along the axis of the first and second connectors, in particular along the axis of the connection system.

### DRAWINGS

Some embodiments and some aspects of the invention will be described below with reference to the attached drawings, provided for illustrative purposes only, wherein:

FIGS. 1 and 2 are a perspective views of the connection system, disposed in a locked condition, according to the present invention;

FIGS. 2A and 2B are a cross section views of the connection system of FIG. 2;

FIG. 3 is an exploded view of the connection system according to the present invention;

FIG. 3A is a cross section view of FIG. 3;

FIG. 4 is a perspective view of the connection system, disposed in an unlocked condition, according to the present invention;

FIG. 4A is a cross section view of FIG. 4;

FIGS. 5 and 6 are different embodiments of a disposable set according to the present invention;

FIG. 7 is a perspective view of a further embodiment of the connection system;

FIGS. 7A and 7B are cross section views of the embodiment of FIG. 7 in an unlocked and in a locked condition respectively;

FIG. 8 is a perspective view of an additional embodiment of the connection system;

FIGS. 8A and 8B are cross section views of the embodiment of FIG. 8 in an unlocked and in a locked condition respectively;

FIG. 9 is a perspective view of another embodiment of the connection system;

FIG. 9A is a cross section view of the embodiment of FIG. 9 in an unlocked condition.

### DEFINITIONS

In this detailed description, corresponding parts illustrated in the various figures are indicated with the same numerical references. The figures may illustrate the invention by means of non-scale representations; therefore, parts and components illustrated in the figures relating to the object of the invention may relate exclusively to schematic representations.

Upstream and/Downstream

The terms upstream and downstream refer to a direction or trajectory of advancement of a fluid configured to flow within the connector or along the fluid line during normal usage of the apparatus.

### Non-Removable

By the wording ‘non-removable’ is meant that the connection system is in a locked condition under normal use. As below explained, in the locked condition of the connection system at least one locking projection 24 of the collar 20 and at least one locking projection 14 of the second connector 11 are in a coupled configuration, and a hooking portion 23 of the collar 20 and a hooking portion 13 of the second connector 11 are in an engaged configuration; in said locked condition the collar 20 is irremovably engaged to the second connector 11 through an engagement rotation and the locking projection 14 and the hooking portion 13 of the second connector 11 respectively cooperating with the locking projection 24 and the hooking portion 23 of the collar 20 prevent disengagement of the first connector 1 from the second connector 11.

### DETAILED DESCRIPTION

### Connection System 100

Reference number 100 is directed to a connection system, as shown in figures from 1 to 4, for a medical apparatus: the connection system 100 may be used to connect, in a fluid tight manner, fluid lines 51 of a medical machine 200, such as a blood treatment apparatus, in particular a dialysis machine, wherein the fluid lines 51 may be a blood circuit 50a of a disposable set 50, an infusion line 54, a monitoring line 55, an access line 52 or a return line 53.

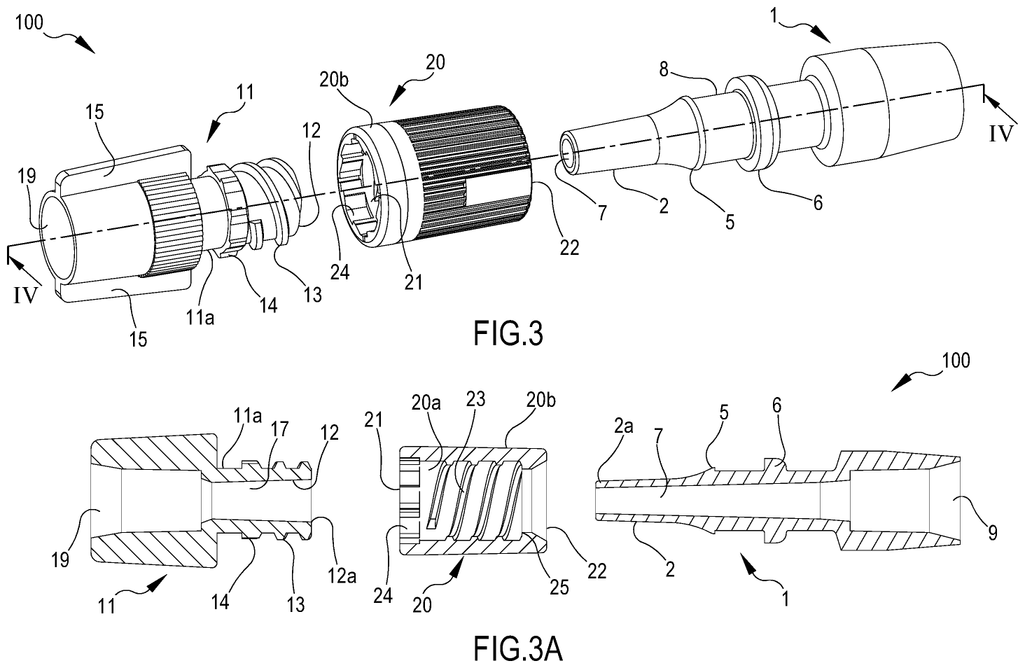

The connection system 100 comprises a first connector 1, shown in the exploded view of FIG. 3 and in the respective cross section in FIG. 3A, comprising a main body having tubular shape and defining an internal channel 7 for fluid passage: the main body of the first connector 1 and the internal channel 7 preferably extend in length along the same fluid flow axis, between a connection port 9 configured to be fixed/connected in a fluid tight manner to a fluid line 51 or directly to the medical machine 200 (or an accessory), and a coupling portion 2. The connection port 9 may be a male or female connection port. Usually, a tube end portion is irremovably fixed inside the connection port via e.g., gluing or bonding. The coupling portion 2 of the first connector 1 has preferably tubular shape and includes a tip portion 2a surrounding the internal channel: the coupling portion 2 has a tapered external surface, so that a diameter of the coupling portion 2 reduces towards the tip portion 2a of the coupling portion 2. In particular, the tapered external surface of the tip portion 2a of the coupling portion 2 has a taper angle comprised between 1° and 6°, more in particular comprised between 3° and 4°, more in detail the taper angle is equal to 3.44°. In an embodiment, the first connector 1, and in particular the coupling portion 2 of the first connector 1, is a Luer type connector: this means that dimensions of the coupling portion 2, the diameter of the internal channel 7 and the overall length of the first connector 1 are consistent with the Luer standards. In a non-limiting embodiment, the first connector 1 has a length comprised between 2 cm and 4 cm, and the internal channel 7 has an average diameter comprises between 2 mm and 3 mm.

The first connector 1 further comprises a main abutment portion 5 arranged on an external lateral wall of the main body, wherein the main abutment portion 5 defines a protrusion radially extending outward from the main body. The abutment portion 5 may have circular shape, for example a ring coupled to the main body extending outwardly. In an embodiment, the first connector 1 may further comprise an auxiliary abutment portion 6 arranged on the external lateral wall of the main body, wherein the auxiliary abutment portion 6 is axially distal from the main abutment portion 5 by an axial gap, so that a recess 8 is defined between the main abutment portion 5 and the auxiliary abutment portion 6. As shown in FIGS. 3 and 3A, the auxiliary abutment portion 6 is axially interposed between the connection port 9 and the main abutment portion 5: similarly, the main abutment portion 5 is interposed between the coupling portion 2 and the auxiliary abutment portion 6.

In the shown embodiment, the first connector 1 is a male connector and it is made in one single piece preferably in plastic material, such as PVC, MBS, PC, or in metallic material, such as stainless steel. The connection system 100 further comprises a second connector 11, shown in the exploded view of FIG. 3 or in the cross section view of FIG. 3A, presenting a main body having tubular shape and defining an internal channel 17 for fluid passage: the main body of the second connector 11 and the internal channel 17 preferably extend in length, along the same fluid flow axis, between a connection port 19 configured to be fixed/connected in a fluid tight manner to a fluid line 51 or directly to the medical machine 200 (or an accessory), and a coupling portion 12. The connection port 19 may be a male or female connection port. Usually, a tube end portion is irremovably fixed inside the connection port 19 via e.g., gluing or bonding. The coupling portion 12 of the second connector 11 is configured to be connected to the coupling portion 2 of the first connector 1 in a fluid tight manner, so that a fluid is able to flow between the first and the second connectors 1, 11, and vice versa, in particular wherein the fluid is able to flow between the connection port 9 of the first connector 1 and the connection port 19 of the second connector 11, and vice versa. When the coupling portion 2 of the first connector 1 is connected to the coupling portion 12 of the second connector 11, the fluid flow axis of the first connector 1 coincides with the fluid flow axis of the second connector 11, and the internal channels 7, 17 respectively of the first and the second connectors 1, 11 define a unique internal channel for fluid passage extending from the connection port 9 of the first connector 1 to the connection port 19 of the second connector 11. The coupling portion 12 of the second connector 11 has preferably tubular shape and includes a terminal portion 12a surrounding the internal channel 17 and having a tapered internal surface, so that a diameter of the tapered internal surface increases towards the terminal portion 12a. According to the disclosed embodiment, the tapered internal surface of the terminal portion 12a of the second connector 11 is counter shaped to the tapered external surface of the tip portion 2a of the first connector 1. In other words, both the coupling portion 2 of the first connector 1 and the coupling portion 12 of the second connector 11 have conical shape. The tip portion 2a of the first connector 1 is configured to enter into and to contact the terminal portion 12a of the second connector 11, to define a fluid tight connection: the tapered external surface is configured to couple with the tapered internal surface to connect the first and the second connectors 1, 11 in a fluid tight manner. In particular, the tapered internal surface of the terminal portion 12a of the coupling portion 2 has a taper angle comprised between 1° and 6°, more in particular comprised between 3° and 4°, more in detail the taper angle is equal to 3.44°. In the specific embodiment, the taper angle of the coupling portion 2 of the first connector 1 is equal to the taper angle of the coupling portion 12 of the second connector 11. In an embodiment, the second connector 11, and in particular the coupling portion 12 of the second connector 11, is a Luer type connector: this means that dimensions of the coupling portion 12, the diameter of the internal channel 17 and the overall length of the second connector 11 are consistent with the Luer standards. In an embodiment, the second connector 11 has a length comprised between 2 cm and 3 cm, and the internal channel 17 has an average diameter comprises between 3 mm and 4 mm.

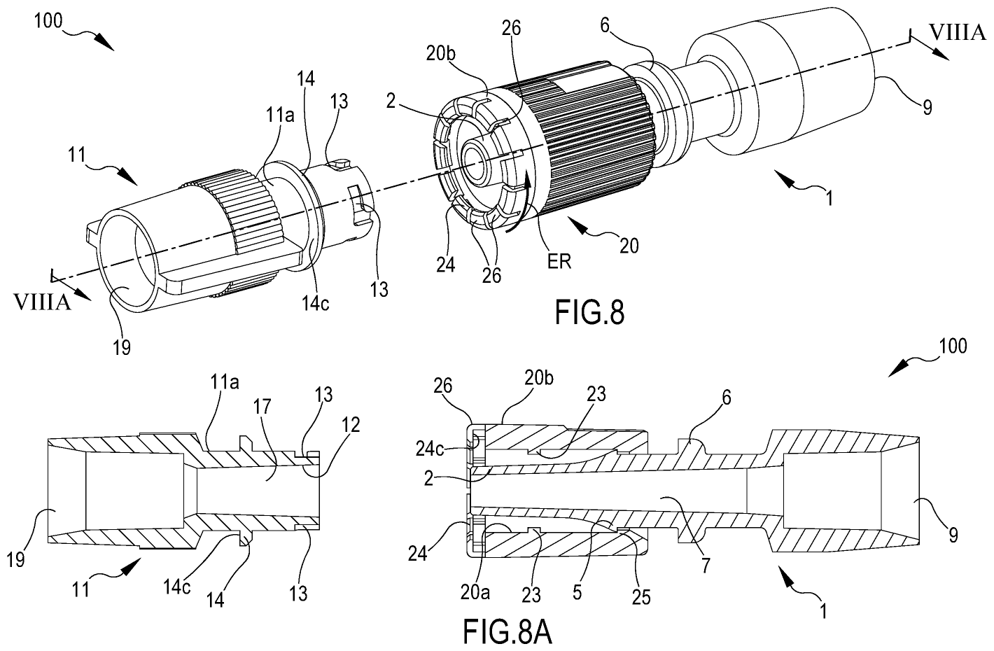

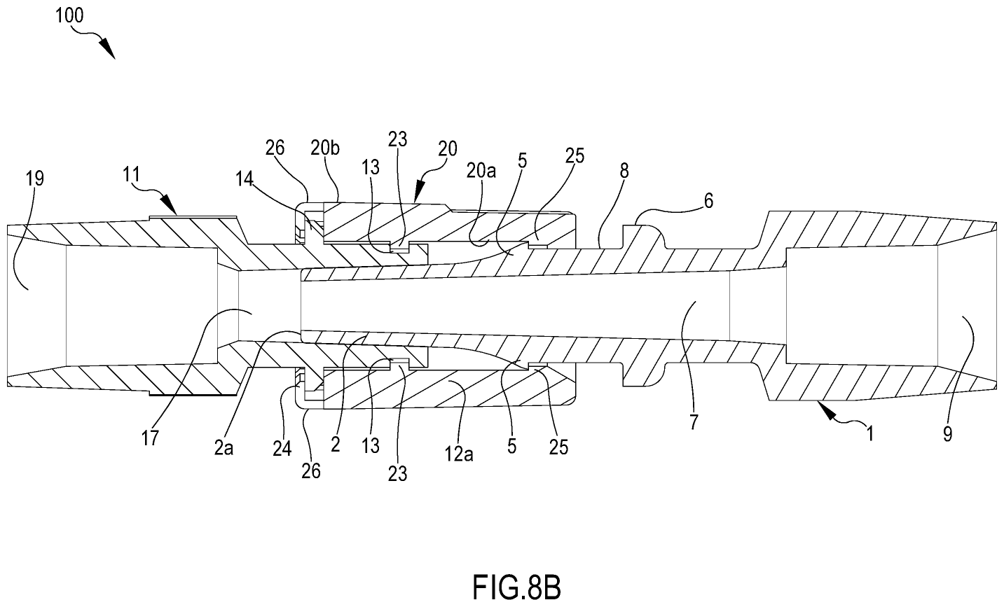

The second connector 11 may further comprise a hooking portion 13 arranged on an external lateral wall 11a of the main body of the second connector 11: in particular the hooking portion 13 may comprise a thread, preferably arranged in the proximity of the coupling portion 12 of the second connector 11. The threads of the hooking portion 13 preferably extend from the terminal end 12a of the second connector 11 and preferably emerge from the external lateral wall 11a of the main body of the second connector 11. In an alternative embodiment, the hooking portion 13 of the second connector 11 may comprise a respective groove or recess to define an undercut joint, such as a bayonet coupling (embodiment shown in FIG. 8). Said groove has a first tract extending substantially parallel to the axis of the second connector, and a second tract extending substantially perpendicular to the first tract. Alternatively said second tract may have arc shape with the concavity facing the terminal end 12a of the second connector 11.

The second connector 11 further comprises at least one locking projection or member 14 arranged on the same external lateral wall 11a of the main body of the second connector 11. In an embodiment, shown in FIG. 3, the at least one locking projection 14 is interposed between the hooking portion 13 and the connection port 19: similarly, the hooking portion 13 is interposed between the at least one locking projection 14 and the terminal end 12a of the second connector 11. The at least one locking projection 14 radially emerges outwardly from the main body of the second connector 11: in particular the at least one locking projection 14 comprises a plurality of socket teeth, wherein the socket teeth are wedge-shaped or have a saw tooth shape. The socket teeth are preferably arranged all around the tubular main body of the second connector 11, as shown in FIG. 3.

In the embodiment, the second connector 11 is a female connector and it is made in one single piece preferably in plastic material, such as PVC, MBS, PC or in metallic material, such as stainless steel. The second connector 11 may also comprise gripping means arranged on the external lateral wall 11a at the connection port 19: the gripping means are configured to allow an operator to grab firmly the second connector 11 especially when connection with the first connector 1 is required. The gripping means 15 preferably comprise tabs, in particular two tabs diametrically opposed, emerging radially outwardly from the main body of the second connector 11.

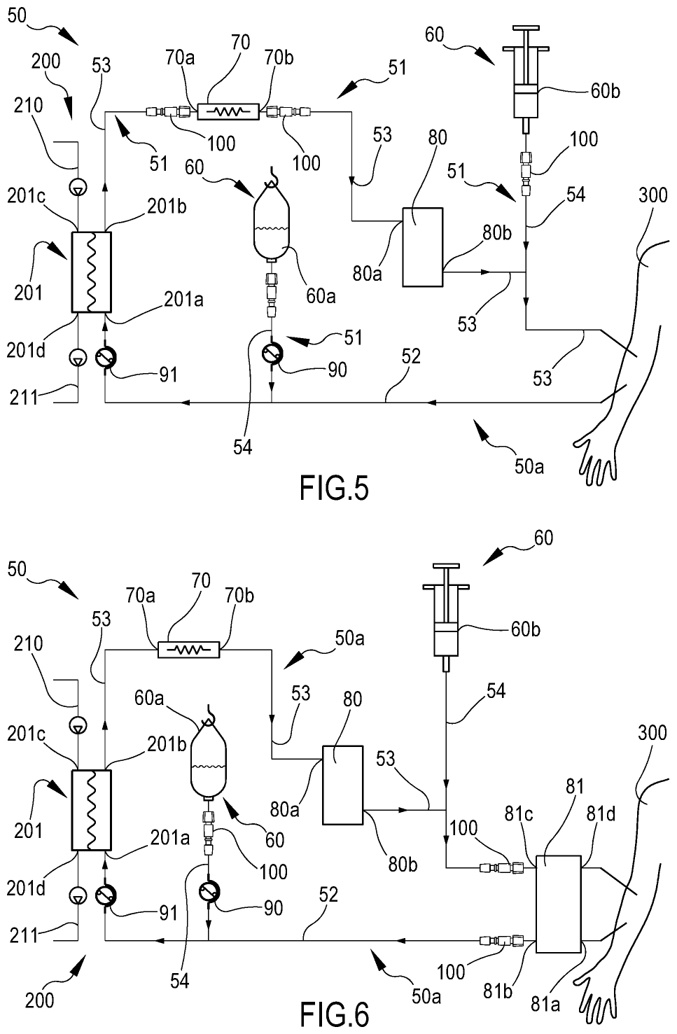

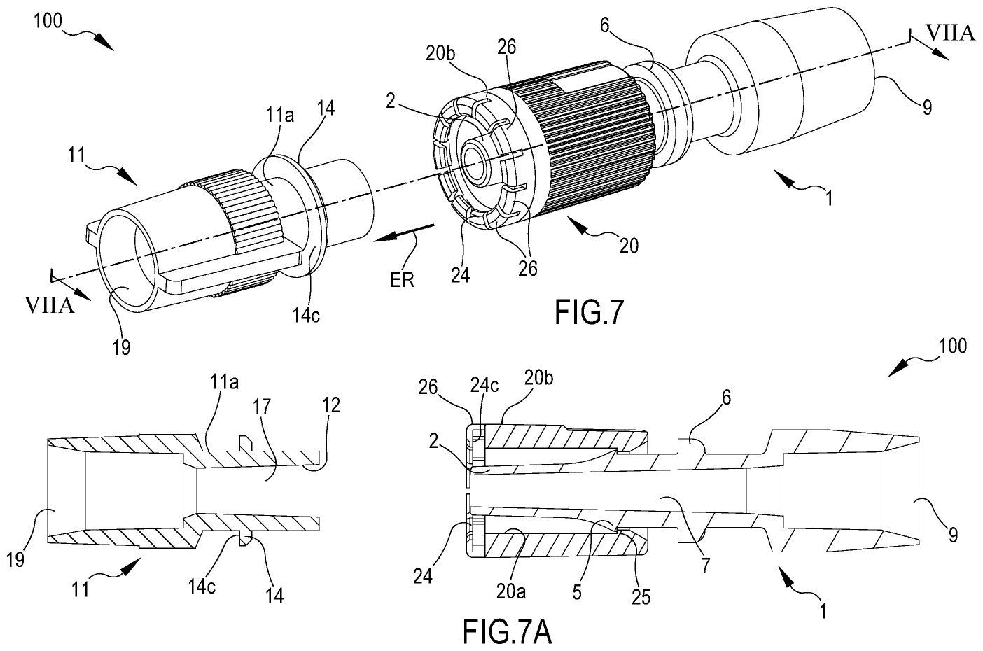

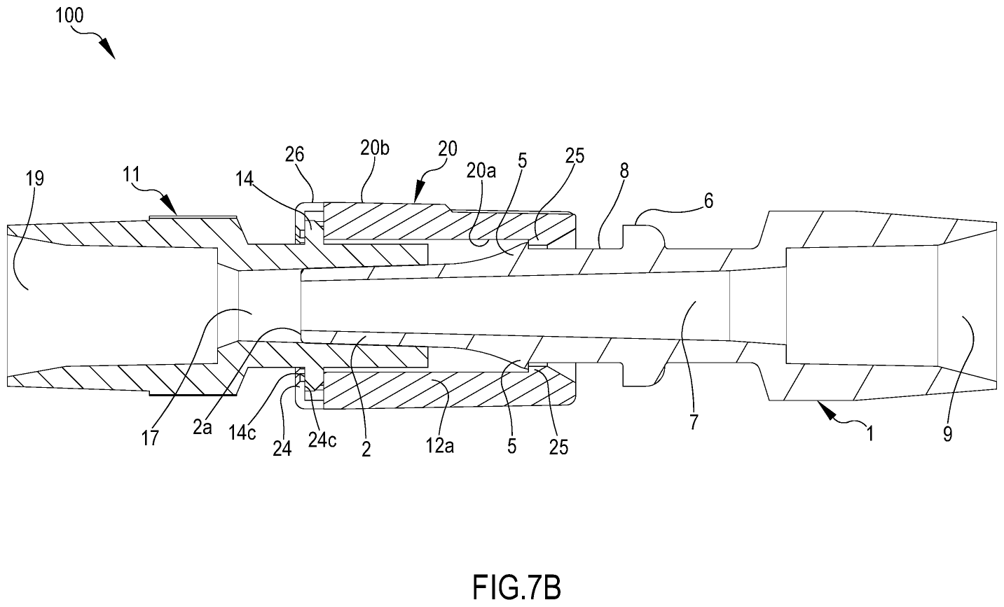

The connection system 100 further comprises a collar 20, shown in the exploded view of FIG. 3 and in the cross section view of FIG. 3A, presenting a main body having tubular shape: in particular the collar extends in length between a first opening 21 and a second opening 22, and the tubular main body comprises an external lateral wall 20b and an internal lateral wall 20a. In an embodiment not shown in the attached figures, the main body of the collar 20 may have polygonal shape, such as squared, rectangular or a combination of polygonal and tubular shapes. The collar 20 comprises an internal channel laterally limited by the first and the second openings 21, 22, wherein the opening 21 is configured to receive through insertion the second connector 11, in particular to receive the coupling portion 12, the hooking portion 13 and the at least one locking projection 14 of the second connector 11, while the second opening 22 allows the collar 20 to be mounted over, and coupled to, the first connector 1, as shown in FIG. 7. In more detail, the second opening 22 receives in insertion the first connector 1, so that the collar surrounds externally at least part of the first connector 1 and the main body of the collar 20 is coaxial with the first connector 1. In particular the collar 20 comprises, preferably at the second opening 22, a respective abutment portion 25 configured to contact the main abutment portion 5 of the first connector 1 when disposed in an abutment configuration, so that the collar 20 is axially constrained with respect to the first connector 1 at least along one axial direction. In particular the collar 20 is axially constrained towards the coupling portion 2 of the first connector 1, so that the collar 20 is prevented to axially move towards the coupling portion 2 of the first connector 1 beyond a certain limit. The abutment portion 25 of the collar 20, as shown in the section of FIG. 6, is arranged at the second opening 22 of the collar 20 and extends radially inwardly.

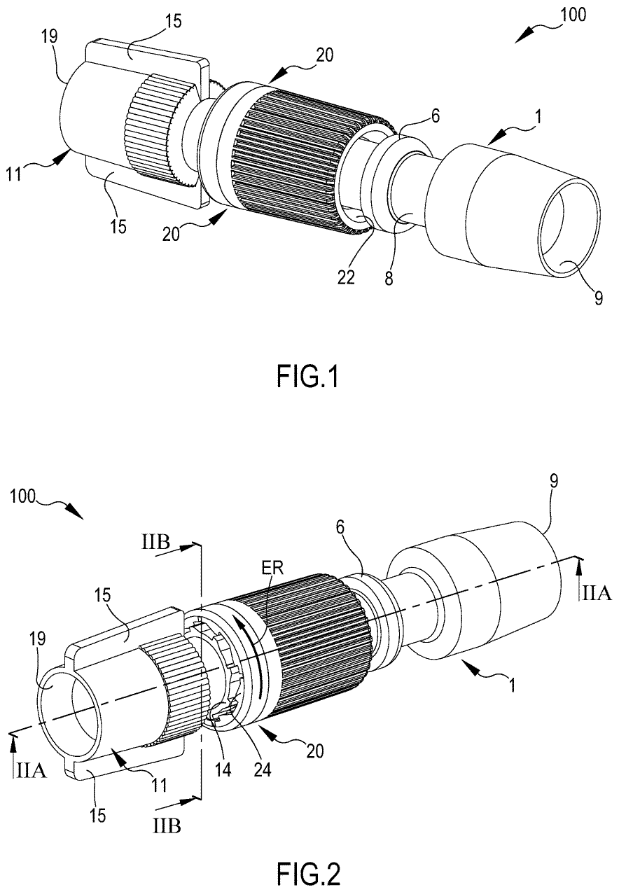

In the disclosed embodiment shown in FIGS. 1 to 4, the first connector comprises both the main and the auxiliary abutment portions 5, 6, so that the abutment portion 25 of the collar is interposed between the main abutment portion 5 and the auxiliary abutment portion 6 of the first connector: therefore, the collar is a axially constrained to the first connector 1 through the main and auxiliary abutment portions 5, 6 of the first connector 1, so that an axial movement of the collar 20 with respect to the first connector 1 is substantially prevented. Alternatively or in addition, the collar 20 may be axially movable with respect to the first connector 1 within the axial gap defined between said main and auxiliary abutment portions 5, 6 of the first connector 1, and axially constrained to the first connector 1 out of this axial gap. In particular, the collar 20 is axially movable with respect to the first connector 1 of an amount proportional to the distance between the main and the auxiliary abutment portions 5, 6 of the first connector 1. The axial movement is defined along the fluid flow axis of the first connector 1 and/or of the second connector 11. Anyhow, if the first connector comprises the main and the auxiliary abutment portions 5, 6, the abutment portion 25 of the collar 20 is arranged at, and in particular surrounds, the recess 8 of the first connector 1.

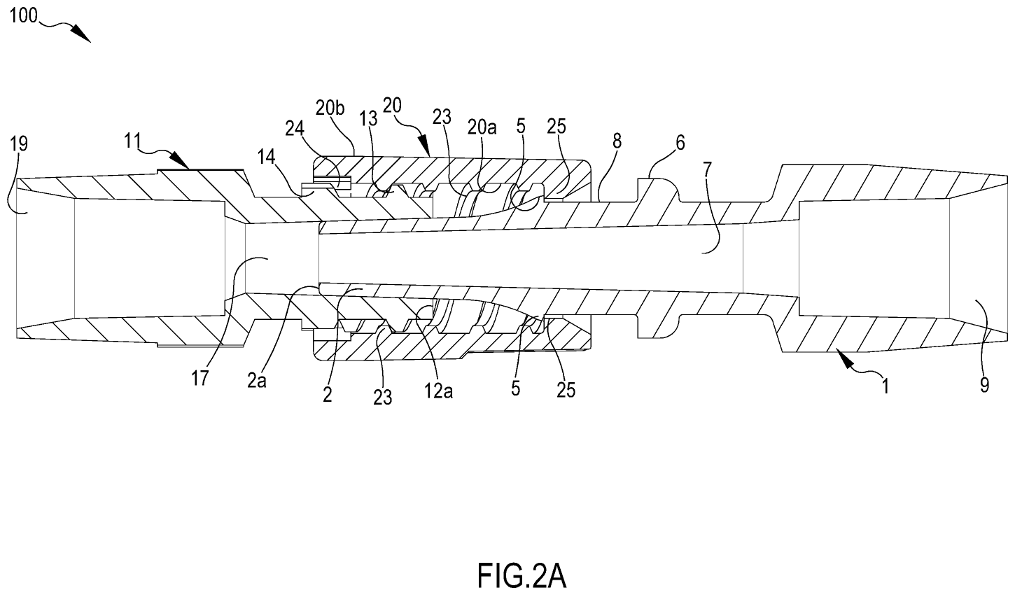

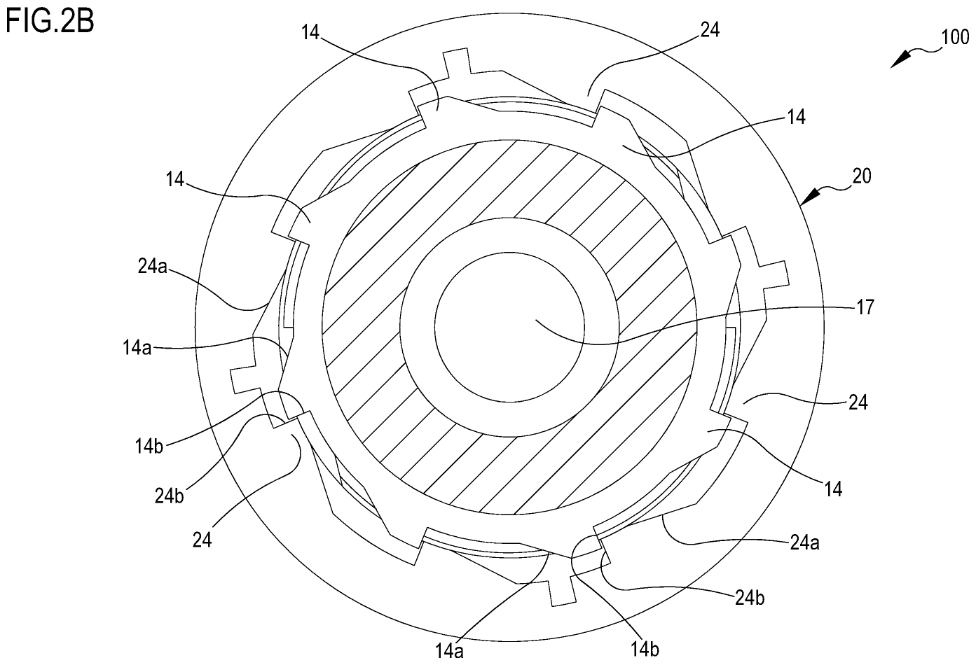

In this specific embodiment, the second opening 22 has an internal diameter lower than an external diameter of the main abutment portion 5 of the first connector 1, preventing in this way the axial movement. In other words, the first connector 1 and the collar, although they are distinct bodies both made in a single piece, are axially coupled each other, being the collar 20 movable by rotation with respect to the first connector 1. In an alternative embodiment not shown in the attached figures, the collar 20 and the first connector 1 are made in one single piece. In an embodiment, the collar 20 has a length, measured along an axis of its tubular main body, comprised between 8 mm and 25 mm, and a diameter comprised between 7 mm and 15 mm. The collar 20 may further comprise a respective hooking portion 23 configured to be engaged with the hooking portion 13 of the second connector 11 through an engagement rotation ER along an engagement direction (see FIG. 2) defining an engaged configuration shown in FIGS. 1 and 2, so that when the connection system 100 is disposed in this engaged configuration the hooking portion 23 of the collar 20 and the hooking portion 13 of the second connector 11 prevent axial removal of the collar 20 from the second connector 11. In an embodiment, the hooking portion 23 of the collar comprises a thread configured to engage the thread of the second connector 11 through this engagement rotation. In an embodiment, the hooking portion 23 is arranged on the internal lateral wall 20a of the collar 20. In other words, in this engaged configuration, the collar 20 is screwed over the second connector 11 through the respective threads, defining an axial constraint between the collar 20 and the second connector 11.

In an alternative embodiment, the hooking portion 23 of the collar 20 comprises an undercut joint (see FIG. 8) and the hooking portion 13 of the second connector 11 comprises the respective undercut joint, wherein the undercut joint of the collar 20 is configured to axially engage the undercut joint of the second connector 11 defining a bayonet coupling for example through the same engagement rotation ER, so that an axial movement, in particular an axial separation, of the first and second connectors 1, 11 is prevented: this axial movement or axial separation is considered along the fluid flow axis of the first or second connectors 1, 11. In this alternative embodiment, the engagement rotation is comprised between 10° and 180°, in particular comprised between 15° and 95°.

Since the collar 20 is axially coupled to the first connector 1, when the connection system 100 is disposed in this engaged configuration the first connector 1 is axially coupled to the second connector 11, so that disconnection of the first and second connectors 1, 11 is prevented. In particular, at an end stage of the engagement rotation, the hooking portions 13, 23 respectively of the second connector 11 and of the collar 20 contribute to tighten the connection and to prevent disconnection between the coupling portion 2 of the first connector 1 and the coupling portion 12 of the second connector 11. During an engagement rotation ER wherein the collar 20 rotates to determine the engagement rotation, the coupling portion 2 of the first connector 1 is allowed to stay aligned with the coupling portion 12 of the second connector 11 due to the fact that the collar 20 is freely movable by rotation with respect to the first connector 1: once the connection between the coupling portion 2 of the first connector 1 and the coupling portion 12 of the second connector 11 is established, the collar 20 is still freely movable by rotation with respect to the first and second connectors 1, 11, so that the subsequent engagement rotation ER does not determined friction between the coupling portion 2 of the first connector 1 and the coupling portion 12 of the second connector 11. In other words and according to an embodiment, during the engagement rotation the first and the second connectors 1, 11 may be aligned each other without reciprocal rotation, while the collar 20 rotates with respect both the first and second connectors 1, 11 to determine engagement of the hooking portions 13, 23.

The hooking portion 23 of the collar 20 may comprise, as shown in FIG. 8, a protrusion radially extending inwardly and configured to insert into the groove of the hooking portion 13 of the second connector. The collar 20 further comprises a respective at least one locking projection 24 engageable to the at least one locking projection 14 of the second connector 11 through the engagement rotation ER of the collar 20 with respect to the second connector 11 along the engagement direction. The connection system 100 may be disposed in a coupled configuration, wherein the at least one locking projection 24 of the collar 20 cooperates with the at least one locking projection 14 of the second connector 11, preventing a disengagement rotation of the collar 20 with respect to the second connector 11: this disengagement rotation is a rotation in a opposite direction with respect to the engagement rotation. The coupled configuration is defined at least at an end stage of the engagement rotation between the collar 20 and the second connector 11, meaning that when the engagement rotation is complete, a disengagement rotation of the collar 20 with respect to the first connector 1 is prevented. In addition, the coupled configuration may be defined at an intermediate or initial stage of the engagement rotation ER: this means that, as soon as the hooking portion 23 of the collar 20 engages the hooking portion 13 of the second connector 11, the locking projections 14, 24, respectively of the second connector 11 and of the collar 20, also cooperate each other, preventing the disengagement rotation.

In an embodiment, this locking projections 24 are arranged on the internal lateral wall 20a of the collar, so that the locking projection 24 and the hooking portion 23 of the collar 20 are arranged side by side. In particular, the at least one locking projection 24 of the collar may comprise a plurality of socket teeth, wherein the socket teeth are wedge-shaped or have a saw tooth shape. The at least one locking projection 14 radially emerges inwardly from the internal lateral wall 20a of the collar, as shown in FIG. 3. When the socket teeth of the collar 20 face, and in particular contact, the socket teeth of the second connector 11, the engagement rotation is allowed and the disengagement rotation is prevented.

The socket teeth of both the second connector 11 and the collar 20 are arranged circumferentially all around the external lateral wall 11a of the second connector 11 and circumferentially all around the internal lateral wall 20a of the collar 20. The socket teeth radially define recesses and protrusions alternately, so that the second connector 11 defines a maximum external diameter of encumbrance measured at the protrusions of the socket teeth, and wherein the collar 20 defines a minimum internal diameter measured at the protrusions of the socket teeth of the collar 20: this maximum external diameter of encumbrance is higher than this minimum internal diameter so that, when the socket teeth of the second connector 11 are faced to or in contact with the socket teeth of the collar 20, the protrusions of the socket teeth of the second connector 11 are placed into the recesses of the socket teeth of the collar 20 and vice versa. This is clearly shown in the cross section view of FIG. 2B, wherein the saw tooth shape of the locking projections allows the engagement rotation and prevents the disengagement rotation.

In an embodiment, the socket teeth of the second connector 11 and of the collar 20 comprise each a respective slide surface 14a, 24a (FIG. 2B) tilted with respect to the external surface of the second connector 11 and to the internal lateral wall 20a of the collar 20: preferably this slide surface 24a is tilted by an angle lower than 60°. Each of the socket teeth of the second connector 11 and of the collar 20 further comprise respective lock surfaces 14b, 24b which are substantially perpendicular with respect to the external surface of the second connector 11 and to the internal lateral wall 20a of the collar 20: the slide surface 14a, 24a and the lock surface 14b, 24b of each socket tooth are connected together at a vertex defining the protrusion. In other words, the slide surface 14a, 24a and the lock surface 14b, 24b, of one socket tooth define a triangle shaped tooth, wherein these teeth are radially arranged over the internal lateral wall 20a of the collar 20 and over the external lateral wall 11a of the second connector 11. The socket teeth of the collar 20 are oriented in an opposite direction with respect to the socket teeth of the second connector 11, so that the lock surfaces 14b of the socket teeth of the second connector 11 may be faced to the lock surfaces 24b of the collar 20. Each slide surface 14a of a socket tooth of the second connector 11 is configured to cooperate or contact with a respective slide surface 24a of a socket tooth of the collar 20, so that the engagement rotation is allowed: on the other hand, each lock surface 14b of a socket tooth of the second connector 11 is configured to contact, when the connection system 100 is disposed in the coupled configuration, with a respective lock surface 24b of a socket tooth of the collar 20, preventing therefore the disengagement rotation: in particular, when the connection system 100 is disposed in the coupled configuration, each lock surface 14b of a socket tooth of the second connector 11 abuts against a respective lock surface 24b of a socket tooth of the collar 20.

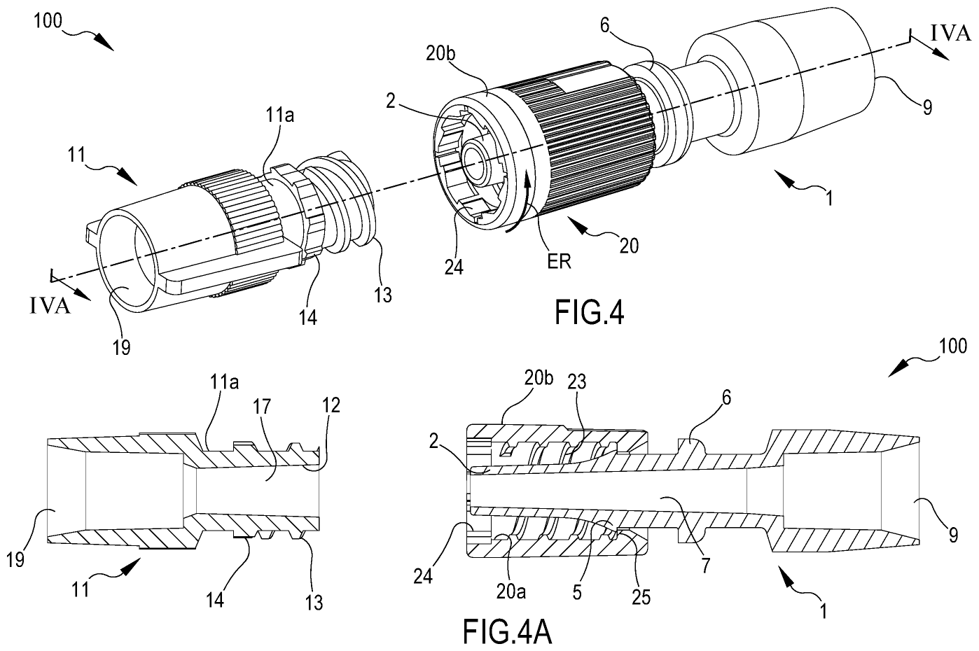

In an embodiment shown in FIGS. 3, 3A, 4 and 4A, the hooking portion 13 of the second connector 11 is interposed between the at least one locking projection 14 and the terminal portion 12a of the coupling portion 12, and the at least one locking projection 24 of the collar 20 is arranged at an inlet of the first opening 21 so that the hooking portion 23 of the collar 20 is interposed between the at least one locking projection 24 and the second opening 22.

In an alternative embodiment not shown in the attached figures, the at least one locking projection 14 of the second connector 11 is interposed between the hooking portion 13 and the terminal portion 12a of the coupling portion 12, and the hooking portion 23 of the collar 20 is arranged at the inlet of the first opening 21 so that the at least one locking projection 24 of the collar 20 is interposed between the hooking portion 23 and the second opening 22 of the collar 20. The connection system 100 is configurable in an unlocked condition, wherein the collar 20 is movable by rotation with respect to the first connector 1 and the at least one locking projection 24 of the collar 20 and the at least one locking projection 14 of the second connector 11 are in an uncoupled configuration wherein the locking projections do not cooperate each other. The unlocked condition is shown in FIG. 4, wherein the first and the second connectors 1, 11 are separated: when the first and the second connectors 1, 11 are separated, the collar 20 is anyhow axially constrained to the first connector through the main and auxiliary abutment portions 5, 6 of the first connector 1 cooperating with the abutment portion 25 of the collar. In addition, the unlocked condition may also be defined when the hooking portion 13 of the second connector 11 is engaged with the hooking portion 23 of the collar 20, which defines the engaged configuration: this means that, when an engagement rotation is at an initial or intermediate stage, the at least one locking projection 24 of the collar 20 and the at least one locking projection 14 of the second connector 11 do not cooperate yet, allowing disengagement rotation of the collar 20 with respect to the first connector 1. The connection system 100 is further configurable in a locked condition, shown in FIGS. 1, 2, and in the section view of FIG. 2A, wherein the at least one locking projection 24 of the collar 20 and the at least one locking projection 14 of the second connector 11 are in the coupled configuration, and the hooking portion 23 of the collar 20 and the hooking portion 13 of the second connector 11 are in the engaged configuration. In this locked condition, the collar 20 is irremovably engaged to the second connector 11 through said engagement rotation: in particular, the at least one locking projection 14 and the hooking portion 13 of the second connector 11 respectively cooperate with the at least one locking projection 24 and the hooking portion 23 of the collar 20 preventing disengagement of the first connector 1 from the second connector 11. Moreover, when the connection system 100 is disposed in the locked condition, the coupling portion 2 of the first connector 1 is connected to and/or inserted into the coupling portion 12 of the second connector 11, so that fluid tightness between the first and the second connector 1, 11 is established. Moreover, in this locked condition, the abutment portion 25 of the collar 20 abuts against the main abutment portion 5 of the first connector 1, preventing the axial movement, and therefore separation, of the first connector with respect to the collar.

In an embodiment, the collar 20, when engaged to the second connector 11 by the respective threads, may present a slight axial movement with respect to the second connector 11, especially at an initial or intermediate stage of the engagement rotation: this may happen if the width of the thread wire of the second connector 11, is lower than the thread pitch of the collar 20, defining a gap in between, which leads to an axial movement of the collar with respect to the second connector. In this case, the locking portions 14 and 24 respectively of the second connector 11 and of the collar 20 should have a width, measured parallel to the fluid flow axis, higher than this gap, in order to prevent decoupling. In the shown embodiment, when the connection system 100 is in the locked condition and/or in the engaged configuration, the fluid flow axis of the first connector 1 coincides with the fluid flow axis of the second connector 11, and the collar is coaxial with this fluid flow axis.

FIGS. 8, 8A, 8B and 7, 7A and 7B show further embodiments of the non-removable connection system wherein the second connector 11 comprises the at least one locking projection 24 and the collar comprises the respective at least one locking projection 14. According to the embodiments shown in FIGS. 7, 7A and 7B, no hooking portions, such as threads or bayonet couplings, are provided on the second connector and on the collar.

The locking projections 14 of the second connector 11 and the locking projections 24 of the collar 20 comprise at least one protrusion respectively extending radially outwardly and inwardly. The protrusions of the collar 20 comprise an abutment portion 24c facing the inner volume of the collar 20, while the protrusions of the second connector 11 comprise an abutment portion 14c facing opposite with respect to the fluid coupling portion 12 of the second connector 11. The abutment portion 24c of the protrusions 24 of the collar 20 are configured to abut against the abutment portion 14c of the protrusions 14 of the second connector 11, as shown in the section of FIGS. 7B and 8B, when the connection system is arranged in the locked condition in order to prevent axial disengagement of the collar from the second connector. The collar 20 and the second connector 11, according to the embodiment of FIGS. 8, 8A and 8B, further comprises the hooking portions 23 and 13: in particular FIG. 8 shows a hooking portion having bayonet coupling. Anyhow, a hooking portion comprising respective threads may also be provided according to this embodiment.

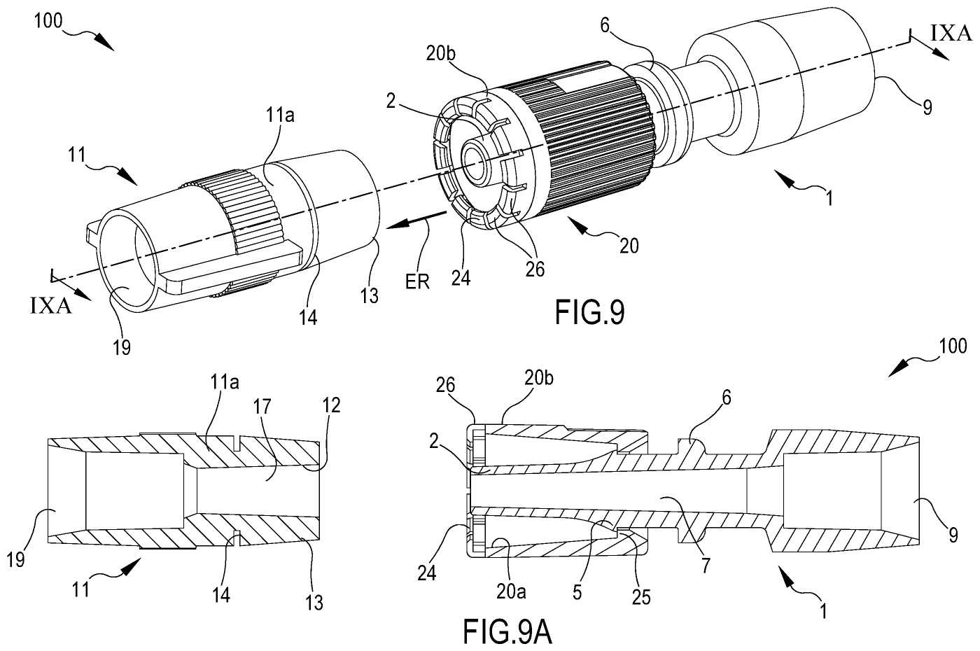

According to the previously embodiments, the locking projections 14, 24 of the second connector and collar comprise protrusions, such as wedge shaped socket teeth: anyhow, as shown in FIGS. 9 and 9A according to a further embodiment, the second connector comprises a locking member 14 having at least one recess, optionally an aperture, and the collar 20 comprises a locking member 24 having at least one protrusion: the protrusions of the collar 20 are configured, in the locked condition, to engage the recess of the second connector 11: in particular the protrusions of the collar 20 are configured to enter at least partially into said recess to define an axial engagement. Therefore, the embodiments shown in FIGS. 1-4A define the coupled configuration and the locked condition by the engagement rotation ER of the collar 20 with respect to the second connector 11: contrary, the embodiments of FIGS. 7 to 9A define the coupled configuration and the locked condition by an axial engagement displacement along the axis of the first and second connector (namely the axis of the connection system). In other terms, the connection system is configurable, through the axial engagement displacement, in a locked condition, wherein the locking projections or members 24 of the collar 20 and the locking projections or members 14 of the second connector 11 are in the coupled configuration, wherein in this locked condition the collar 20 is irremovably engaged to the second connector 11 through the axial engagement displacement, and the locking projections or members 14 of the second connector 11 cooperate with the locking projections or members 24 to prevent disengagement, in particular axial disengagement, of the first connector 1 from the second connector 11. The axial engagement displacement is a displacement wherein the collar and the second connector are approached each other and axially coupled. According to the embodiments of FIGS. 7, 7A, 7B and 9, the collar 20 may freely rotate with respect to the second connector 11 when arranged in the locked condition, preventing at the same time axial disengagement between the collar and the second connector, and therefore preventing axial disengagement between the first and the second connector. Notably, the coupling portions 2 and 12 of the first and second connectors 1, 11 abut each other to define a fluid tight coupling when the connection system is arranged in the locked condition.

According to the above description, it is to be understood that the invention is not supposed to be limited to the disclosed embodiments, but on the contrary, is intended to cover various modifications and equivalent arrangements included within the spirit and the scope of the appended claims.

### Disposable Set 50

The invention also refers to a disposable set 50, shown in FIGS. 5 and 6, for a blood treatment apparatus 200, such as a dialysis machine. The disposable set 50 comprises a plurality of fluid lines 51, such as a blood circuit 50a which includes an access line 52 configured to be connected to a patient 300 and to an inlet port 201a of a blood treatment unit 201, such as a hemofilter, an ultrafilter, a hemodiafilter, a dialyzer, a plasmafilter and the like. The blood circuit 50a also comprises a return line 53 configured to be connected to the patient 300 and to an outlet port 201b of the same blood treatment unit 201. In this configuration, blood is supposed to flow from the patient along the access line 52, through the blood treatment unit 201, and towards the return line 53, as shown by the arrows depicted on the fluid lines of FIGS. 5 and 6. The disposable set 50 may also comprise one or more infusion lines 54 connected to the blood circuit 52 and/or configured to be connected to the patient 300. The infusion line 54 may comprise a container 60 connected to one end of the infusion line and wherein the other end of the infusion line 54 is connected to the blood circuit or to the patient. This container 60 is configured for housing an infusion fluid and is made, for example, of plastic material for medical applications: in particular the container 60 may be a citrate solution container 60a containing a citrate concentrated solution, or calcium solution container 60b containing a calcium concentrated solution, or a substitute fluid solution to be injected into the blood circuit during treatment. FIGS. 5 and 6 shows a (pre-) infusion line 54 arranged upstream with respect to the blood treatment unit 201 and connected to a citrate solution container 60a. The infusion line 54 is also placed upstream a blood pump 91 to provide regional anticoagulation to the extracorporeal blood. At least one pump 90 may be associated to the infusion line 54 and configured to determine fluid flow from the container 60 towards the other end of the infusion line 54, for example towards the access line 52 or to the return line 53. In more detail, the pumps 90 and 91 are peristaltic pumps. Other infusion lines may be included in the circuit and are not represented. For example a pre-infusion line injecting substitution fluid upstream the blood treatment unit 201 and downstream the blood pump 91 may be included, as well as a post-infusion line injecting a substitute solution into the return line 53, for example into the venous bubble trap (not shown). FIGS. 5 and 6 also show an additional infusion line 54 arranged downstream with respect to the to the blood treatment unit 201 and connected to the calcium solution container 60a, for example a syringe configured for housing a calcium solution. This infusion line is intended to reestablish the ion balance in a regional anticoagulation procedure. The blood pump 91 is associated to the access line 52 and/or to the return line 53 to determine blood flow through the treatment unit 201. The disposable set 50 may also comprise a monitoring line 55, not shown in the attached figures, connected to the blood circuit 50a or to any of the infusion lines 54, and configured e.g., to be connected to a pressure or to temperature gauge for parameter detection or to be used as a site access for blood sampling. The disposable set 50 may also comprise a warmer accessory 70, such as a bag or a line and schematically shown in FIGS. 5 and 6, connected to the blood circuit 50a (e.g. downstream the treatment unit 201) and configured to warm up the blood before return to the patient 300. In particular the warmer accessory 70 is connected in series to the return line 53, so that an inlet 70a of the warmer accessory 70 is connected to an upstream tract of the return line 53, and an outlet 70b of the warmer accessory 70 is connected to a downstream tract of the return line 53.

The disposable set 50 further comprises at least one connection system 100 as previously described, which may be connected to the access line 52, the return line 53, to the infusion line 54 and the monitoring line 55. In FIG. 5 the connection system 100 connects the (calcium) infusion line 54 to the blood circuit; alternatively or in addition the connection system may be used to connect the citrate solution container 60a to the pre blood pump infusion line 54. Moreover, the connections system 100 may connect the inlet 70a and the outlet 70b of the warmer accessory 70 to the return line 53, in order to prevent any undesired disconnection. The disposable set 50 also comprises the blood treatment unit 201 having a blood inlet 201a connected to the access line 52, a blood outlet 201b connected to the return line 53, a fluid inlet 201c connected to a fluid delivery line 210 configured to be connected to a source of fresh treatment fluid (not shown in the attached figures), and a fluid outlet 201d connected to a fluid output line 211 configured to be attached to an exhaust unit, such as a discharge bag. In an embodiment not shown in the attached figures, the connection system 100 may also be used to connected the blood inlet 201a of the blood treatment unit 201 to the access line 52, the blood outlet 201b to the return line 53, the fluid inlet 201c to the fluid delivery line 210 and the fluid outlet 201d to the fluid output line 211 (if proper or necessary). The disposable set 50 may also comprise a disposable accessory 80, such as a gas exchanger, an oxygenator or an auxiliary fluid arrangement, connected to the blood line 50a, in particular a gas exchanger 80 may be connected in series to the return line 53. The gas exchanger 80 is configured to remove CO₂ from the blood before returning it to the patient 300. The gas exchanger comprises a blood inlet 80a and a blood outlet 80b for connection to the return line 53: the connection system 100, in an embodiment not shown in the attached figures, may further be used for connecting the blood inlet 80a of the gas exchanger 80 to an upstream tract of the return line 53, and the blood outlet 80b to a downstream tract of the return line 53. The disposable set may also comprise an add-on accessory 81 connected to the blood circuit 50a through the connection system 100 as shown in FIG. 6. The add-on accessory 81 is interposed between the patient (in particular the patient vascular access) and the access and return lines of the blood circuit; the add-on accessory 81 comprises an arterial blood inlet 81a configured to be connected to an arterial access of the patient 300, an arterial blood outlet 81b connected to the access line 52 of the disposable set 50, a venous blood inlet 81c connected to the return line 53 of the disposable set 50, and a venous blood outlet 81d configured to be connected to a venous access of the patient 300. The connection system may be interposed in connection between the arterial blood outlet 81b and the access line 52, and between the venous blood inlet 81c and the return line 53. Obviously, the disposable set may include several additional elements not represented/described since well-known to the skilled person, such as deaeration chambers, pressure monitoring components, infusion or sampling sites, bubble detectors, venous and arterial clamps to stop blood circulation, etc. . . . . The disposable set may be a set for acute or chronic treatments and may assume different configurations with respect to the shown exemplary set. While the invention has been described in connection with what is presently considered to be the most practical and preferred embodiment, it is to be understood that the invention is not to be limited to the disclosed embodiment, but on the contrary, is intended to cover various modifications and equivalent arrangements included within the scope of the appended claims.

1-19. (canceled)

20. A non-removable connection system for a medical apparatus, said non-removable connection system comprising:
a first connector comprising a first fluid coupling portion;
a second connector comprising a second fluid coupling portion connectable, in a fluid tight manner, to the first fluid coupling portion of the first connector, so that a fluid is able to internally flow between the first connector and the second connector, the second connector further comprising a second hooking portion and at least one second locking projection;
a collar coupled to the first connector and comprising a first hooking portion engageable to the second hooking portion of the second connector through an engagement rotation along an engagement direction, wherein in an engaged configuration the first hooking portion of the collar and the second hooking portion of the second connector prevent axial removal of the collar from the second connector;
wherein the collar further comprises at least one first locking projection engageable to the at least one second locking projection of the second connector through the engagement rotation of the collar with respect to the second connector along the engagement direction, wherein in a coupled configuration the at least one first locking projection and the at least one second locking projection prevent a disengagement rotation of the collar with respect to the second connector, said disengagement rotation being a rotation in a opposite direction with respect to said engagement rotation;
wherein by the engagement rotation, the non-removable connection system is configurable at least from an unlocked condition to a locked condition;
wherein in the unlocked condition: (i) the collar is movable by rotation with respect to the first connector and (ii) the at least one first locking projection and the at least one second locking projection are in an uncoupled configuration; and
wherein in the locked condition: (i) the at least one first locking projection and the at least one second locking projection are in the coupled configuration, (ii) the first hooking portion and the second hooking portion are in the engaged configuration, and (iii) the collar is irremovably engaged to the second connector through said engagement rotation and the at least one second locking projection and the second hooking portion respectively cooperating with the at least one first locking projection and the first hooking portion thereby preventing disengagement of the first connector from the second connector.

21. The non-removable connection system of claim 20, wherein:
the first coupling portion of the first connector extends in length along a first fluid flow axis;
the second coupling portion of the second connector extends in length along a second fluid flow axis;
wherein the first fluid flow axis is aligned, at least when the first coupling portion is connected to the second coupling portion, to the second fluid flow axis; and
wherein the engaged configuration determines an axial constraint between the first and second connectors, said axial constraint being along the first fluid flow axis or the second fluid flow axis.

22. The non-removable connection system of claim 20, wherein the first connector comprises a main abutment portion and the collar comprises a respective abutment portion configured to contact the main abutment portion of the first connector when disposed in an abutment configuration, wherein in said abutment configuration the collar is axially constrained with respect to the first connector at least along one axial direction, so that, at least when the non-removable connection system is disposed in the engaged configuration, the collar axially engages the first connector to the second connector, and
wherein the coupled configuration is defined at least at an end stage of the engagement rotation between the collar and the second connector.

23. The non-removable connection system of claim 22, wherein the first connector comprises an auxiliary abutment portion, said auxiliary abutment portion being configured to block an axial sliding of the collar along a direction opposite to said axial direction, and wherein the collar is:
axially constrained to the first connector through said main and auxiliary abutment portions of the first connector, so that an axial movement of the collar with respect to the first connector is substantially prevented, or
axially movable with respect to the first connector within an axial gap defined between said main and auxiliary abutment portions of the first connector, and axially constrained to the first connector out of said axial gap, said axial movement being along the first fluid flow axis of the first connector or the second fluid flow axis of the second connector.

24. The non-removable connection system of claim 22, wherein the first connector comprises a main body having a tubular shape defining an internal channel for passage of the fluid, said main and auxiliary abutment portions radially extending away from said main body, the first connector further comprising a recess interposed between the main and the auxiliary abutment portions,
wherein the abutment portion of the collar is arranged between the main and the auxiliary abutment portions of the first connector into said recess, and
wherein the collar is axially movable with respect to the first connector by an amount proportional to the distance between the main and the auxiliary abutment portions of the first connector.

25. The non-removable connection system of claim 20, wherein the first fluid coupling portion of the first connector includes a tip portion defining an internal channel for fluid transport, said tip portion having a tapered external surface,
wherein the second fluid coupling portion of the second connector includes a terminal portion defining an internal channel for fluid transport, said terminal portion of the second connector having a tapered internal surface counter shaped to the tapered external surface of the tip portion of the first connector, and wherein at least part of the tip portion of the first connector is configured to enter into the terminal portion of the second connector, wherein said tapered external surface coupling to said tapered internal surface connects the first and second connectors in a fluid tight manner.

26. The non-removable connection system of claim 20, wherein the collar comprises a main body having a tubular shape extending between a first opening and a second opening,
wherein the first opening is configured to receive the second coupling portion, the second hooking portion and the at least one second locking projection of the second connector,
wherein the second opening is configured to receive the first connector, wherein when the first connector is inserted within the second opening, the second opening is arranged at a recess of the first connector to allow coupling and an abutment portion of the collar that extends radially inwardly is arranged at the second opening, and
wherein the collar externally surrounds at least part of the first connector and the main body of the collar is coaxial with the first connector.

27. The non-removable connection system of claim 26, wherein:
said at least first one locking projection of the collar is arranged at an inlet of the first opening, said first hooking portion of the collar is interposed between said at least one second locking projection and the second opening of the collar; and
the second hooking portion of the second connector is interposed between the at least one second locking projection and a terminal portion of the second coupling portion of the second connector;
wherein the first hooking portion of the collar and the second hooking portion of the second connector both comprise:
respective threads configured to engage each other, through the engagement rotation, to define the engaged configuration, or
respective undercut joints configured to engage each other as a bayonet coupling, through the engagement rotation, to define the engaged configuration.

28. The non-removable connection system of claim 20, wherein the collar comprises a main body having a tubular shape extending in depth between an internal lateral wall and an external lateral wall, wherein the collar is mounted over the first connector so that the main body externally surrounds at least part of the first connector, wherein the first hooking portion of the collar is arranged on said internal lateral wall of the collar,
wherein the at least one first locking projection of the collar is radially arranged on the same internal lateral wall of the collar, and
wherein the first hooking portion of the collar is side by side with said at least one first locking projection of the collar.

29. The non-removable connection system of claim 20, wherein the second connector has a tubular shape defining an internal channel for fluid transport, said tubular shape of the second connector comprising an external lateral wall,
wherein the second hooking portion of the second connector is arranged on said external lateral wall of the second connector, wherein the second hooking portion is arranged at the second coupling portion of the second connector, and
wherein the at least one second locking projection of the second connector is radially arranged on said external lateral wall of the second connector, wherein the second hooking portion of the second connector is side by side with said at least one second locking projection of the second connector.

30. The non-removable connection system of claim 20, wherein both the at least one second locking projection of the second connector and the at least one first locking projection of the collar comprise respectively a plurality of socket teeth, so that when the socket teeth of the collar face the socket teeth of the second connector, the engagement rotation is allowed and the disengagement rotation is prevented.

31. The non-removable connection system of claim 30, wherein the socket teeth of both the second connector and the collar radially define recesses and protrusions alternately, wherein the second connector defines a maximum external diameter of encumbrance measured at the protrusions of the socket teeth, wherein the collar has a tubular shape and presents a minimum internal diameter measured at the protrusions of the socket teeth of the collar, wherein the maximum external diameter of encumbrance is higher than said minimum internal diameter so that, when the socket teeth of the second connector are in contact with the socket teeth of the collar, the protrusions of the socket teeth of the second connector are inserted into the recesses of the socket teeth of the collar and vice versa.

32. The non-removable connection of claim 20, wherein the first connector and the collar are two separate pieces that may be subsequently axially coupled, the collar being free to rotate with respect to the first connector at least when the second connector is not coupled to the collar and to the first connector and wherein, when the non-removable connection system is disposed in the engaged configuration, at least part of the second coupling portion of the second connector is interposed between the collar and at least part of the first coupling portion of the first connector.

33. The non-removable connection system of claim 20, comprising a fluid line of a medical machine, wherein
the first connector extends in length between the first coupling portion and a first connection port, wherein the first connection port is fixed to an end of said fluid line of the medical machine; and
the second connector extends in length between the second coupling portion and a second connection port, wherein the second connection port is fixed to an end of said fluid line of the medical machine.

34. A non-removable connection system for a medical apparatus, said non-removable connection system comprising:
a first connector comprising a first fluid coupling portion;
a second connector comprising a second fluid coupling portion connectable, in a fluid tight manner, to the first fluid coupling portion, so that a fluid is able to internally flow between the first and second connectors, the second connector further comprising at least second one locking member;
a collar coupled to the first connector and comprising at least one first locking member engageable to the at least one second locking member through an axial engagement displacement along an engagement direction, wherein in a coupled configuration the at least one first locking member and the at least one second locking member prevent an axial disengagement displacement of the collar with respect to the second connector;
said axial disengagement displacement being a displacement in an opposite direction with respect to said axial engagement displacement;
and wherein the non-removable connection system is configurable, through the axial engagement displacement, from an unlocked condition to a locked condition;
wherein in the unlocked condition the collar, the at least one first locking member of the collar and the at least one second locking member of the second connector are in an uncoupled configuration; and
wherein in the locked condition: (i) the at least one first locking member of the collar and the at least one second locking member of the second connector are in the coupled configuration, and (ii) the collar is irremovably engaged to the second connector through said axial engagement displacement, and the at least one second locking member of the second connector cooperates with the at least one first locking member thereby preventing disengagement of the first connector from the second connector.

35. The non-removable connection system of claim 34, wherein the axial engagement displacement is a displacement directed parallel to an axis of the first and second connectors, said axial engagement displacement being a displacement directed along the axis of the first and second connectors and along the axis of the non-removable connection system.

36. The non-removable connection system of claim 34, wherein both the at least one second locking member of the second connector and the at least one first locking member of the collar comprise at least one protrusion radially extending outwardly and inwardly respectively, wherein the protrusions of the collar comprise an abutment portion facing an inner volume of the collar and the protrusions of the second connector comprise an abutment portion facing opposite with respect to the second fluid coupling portion of the second connector, the abutment portion of the protrusions of the collar abutting against the abutment portion of the protrusions of the second connector when the non-removable connection system is arranged in the locked condition to prevent axial disengagement of the collar from the second connector.

37. The non-removable connection system of claim 34, wherein the second connector comprises a second hooking portion, and the collar comprises a first hooking portion engageable to the second hooking portion through an engagement rotation along an engagement direction, wherein in an engaged configuration the first hooking portion and the second hooking portion prevent axial removal of the collar from the second connector.

38. A disposable set for a blood treatment apparatus comprising:
a blood circuit including:
an access line configured to be connected to a patient and to an inlet port of a blood treatment unit;
a return line configured to be connected to the patient and to an outlet port of the blood treatment unit;
an infusion line connected to the blood circuit or configured to be connected to the patient; and
a monitoring line connected to the blood circuit or to the infusion line,
wherein the non-removable connection system of claim 20 is connected to at least one of the access line, the return line, the infusion line and the monitoring line.
